(19) United States
(12) Patent Application Publication
(10) Pub. No.: US 2013/0317401 A1
(43) Pub. Date: Nov. 28, 2013

(54) SHOULDER ABDUCTION SLING-PILLOW-IMMOBILIZER SYSTEM

(71) Applicant: FanCastic Products, Inc., San Diego, CA (US)

(72) Inventor: Mandy Joslin, San Diego, CA (US)

(73) Assignee: FanCastic Products, Inc., San Diego, CA (US)

(21) Appl. No.: 13/902,736

(22) Filed: May 24, 2013

Related U.S. Application Data
(60) Provisional application No. 61/651,521, filed on May 24, 2012.

(51) Int. Cl. A61F 5/37

(52) U.S. Cl. CPC A61F 5/3738
USPC 602/4

(57) ABSTRACT

A kit for supporting a patient's arm includes a waist band, to be worn around the patient's torso and having a first fastener; a pillow having second and third fasteners; and an arm sling with a fourth fastener attached to at least one of the panels of the arm pouch. The first and second fasteners are fastenable to and releasable from each other to releasably secure the pillow to the waist band, and the third and fourth fasteners are fastenable to and releasable from each other to releasably secure the sling to the pillow, to thereby secure the arm at an abducted position relative to the torso. The first and fourth fasteners may also be fastenable to and releasable from each other to releasably secure the sling to the waist band when the pillow is not present, to immobilize the arm at a non-abducted position.

### CROSS-REFERENCES TO RELATED APPLICATIONS

This application claims the benefit of U.S. Provisional Application No. 61/651,521, filed on May 24, 2012, the entirety of which is incorporated by reference herein.

STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Physicians frequently treat many shoulder ailments with shoulder slings. For example, following a shoulder dislocation or shoulder surgery, a physician may place the patient's affected arm in a sling so that the shoulder remains motionless while it heals. Many simple slings comprise just a pouch that is supported by a strap around the wearer's neck. The wearer's forearm rests within the pouch such that the elbow is at a 90 degree angle and the forearm extends across the wearer's abdomen. However, a sling alone does not immobilize the arm to the extent necessary to heal from many procedures.

For certain shoulder treatments or at certain times during the healing process it is sufficient or even advantageous for the aim to be immobilized immediately against the front of the body, but for other treatments it is advantageous for the patient to maintain his or her shoulder at certain angles of abduction, extension, and rotation. For these treatment situations, a physician frequently employs a sling in combination with an elevation support device, such as a pillow or bolster. The pillow maintains the wearer's arm at the desired angles, while straps and/or a pouch maintain the position of the wearer's arm relative to the pillow. Such a pillow is typically either integral to a sling, or provided completely separate from it. In the latter case, a number of straps, buckles, or the like are necessary to retain the pillow in the desired position relative to the body, and the arm in the desired position with respect to the pillow.

### BRIEF SUMMARY OF THE INVENTION

The present disclosure thus provides a kit including a combination of sling, pillow, and waist band, all of which are provided separately and can be removably secured to one another with simple hook and loop fasteners (commonly known as Velcro) to create a customized system almost entirely free of plastic or metal buckles. The waist band can be fastened to itself to secure it around the patient's waist. Hook tape is provided along a portion of outside surfaces of both the waist band and the pillow, and interior surfaces of the pillow and sling include loop fasteners, so that the sling can be attached to the waist band with or without the intervening pillow. Additional straps may be provided to help immobilize the sling (and thus the arm) with respect to the pillow or waist band (and thus the body).

In more detail, exemplary embodiments of the present disclosure provide a kit for supporting a patient's arm. The kit includes a waist band, to be worn around the patient's torso and having a first fastener; a pillow having a second and a third fastener; and a sling having two panels defining a pouch to support the arm, a strap attached to the panels to be hung from the patient's opposite shoulder, and a fourth fastener attached to at least one of the panels. The first and second fasteners are readily repeatedly fastenable to and releasable from each other to releasably secure the pillow to the waist band, and the third and fourth fasteners are readily repeatedly fastenable to and releasable from each other to releasably secure the sling to the pillow, to thereby secure the arm at an abducted position relative to the torso.

The first and fourth fasteners may also be readily repeatedly fastenable to and releasable from each other to releasably secure the sling to the waist band when the pillow is not present, to immobilize the arm at a non-abducted position.

The fasteners may be hook and loop fasteners.

The kit may further include an additional strap to wrap around the sling and the arm, and at least partway around the pillow, when the pillow is present. The additional strap may also be wrappable around the sling, the arm, and the waistband, when the pillow is not present. The additional strap may be readily repeatedly fastenable to and releasable from either of these wrapped positions. The additional strap may have a first end with a fifth fastener and a second end with a sixth fastener The fifth and sixth fasteners may be fastenable to the pillow or to the waistband to secure the additional strap to either of the wrapped positions. The fifth and sixth fasteners may be hook and loop fasteners.

Another embodiment of an additional strap is to be wrapped behind the patient's elbow. This additional strap has a first end readily repeatedly fastenable to and releasable from the sling. A second end of the additional strap is readily repeatedly fastenable to and releasable from the waist band, when the pillow is not present; or the pillow, when the pillow is present, in a position at which the additional strap wraps behind the patient's elbow, whether or not the pillow is present. The first and second ends of the additional strap may include hook and loop fasteners.

### BRIEF DESCRIPTION OF THE DRAWINGS

FIG. 1 is a perspective view of a sling.

FIG. 2 is a perspective view of a waist band.

FIG. 3 is a perspective view of a pillow.

FIG. 4 is a front view of a forearm strap.

FIG. 5 is a front view of an elbow strap.

FIGS. 6-14 illustrate a method of use of the inventive system, where:

FIG. 6 illustrates the application of a waistband.

FIG. 7 illustrates the application of a pillow.

FIG. 8 illustrates the application of a sling when the pillow is used.

FIG. 9 illustrates the attachment of the sling to the pillow.

FIG. 10 illustrates the application of the forearm strap when the pillow is used.

FIG. 10A illustrates attachment of the forearm strap to the pillow, with the arm and sling removed for clarity.

FIG. 11 illustrates the application of the sling when the pillow is not used.

FIG. 12 illustrates the attachment of the sling to the waist band, when the pillow is not used, and the optional application of the forearm strap.

FIG. 13 illustrates the application of the forearm strap when the pillow is not used.

FIG. 14 illustrates the application of the elbow strap when the pillow is not used.

### DETAILED DESCRIPTION OF THE INVENTION

Post shoulder surgery products are notoriously cheap; ill fitting; uncomfortable for the patient; and nearly impossible, given the myriad of strap devices, antiquated buckles, and lack of precise sizing options, for a healthcare worker to apply them correctly to the patient. The system described herein speaks to that frustration by providing a sleek kit of easily used elements, dually usable with or without an abduction pillow. The system is easily and comfortably worn by the patient and easily applied by the healthcare worker.

Furthermore, the system described herein is the only such system that is usable with the ultra-comfortable Ultimate Arm Sling®, whose stretch element does not fit the prototype of other known shoulder immobilization systems. This system was developed after years of requests from orthopedic surgeons who use the comfortable, stretchable Ultimate Arm Sling® and were frustrated with the lack of shoulder immobilizer systems that could be used with such a sling.

Turning now to the Figures, an exemplary embodiment of such a system will be described in detail.

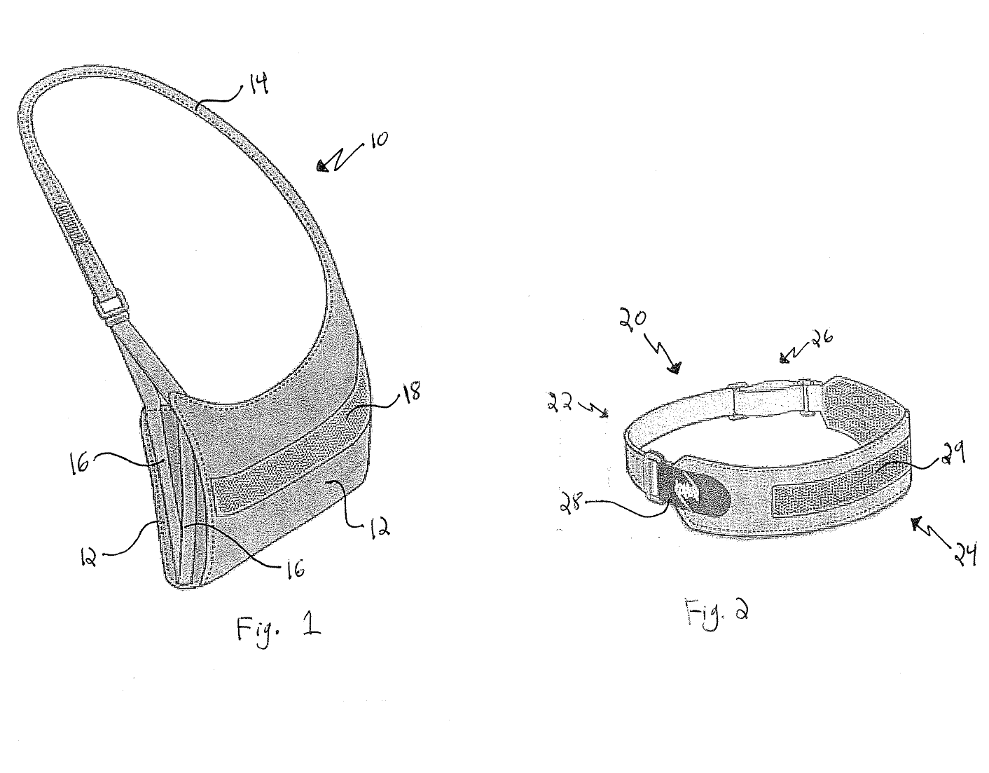

FIG. 1 illustrates a sling 10. The sling 10 may be of any desired construction. In a presently preferred embodiment, the sling 10 is the Ultimate Arm Sling® available from Joslin Orthopedic Gear and described in U.S. Pat. Nos. 6,770,044, 6,102,877, and 5,792,083, the disclosures of all of which are hereby incorporated by reference in their entirety, but the sling may be of any desired construction. As is generally known in the art, the sling 10 has two panels 12 defining a pouch dimensioned for an arm to rest therein, and a strap 14 to support the arm by being hung on the patient's non-treatment shoulder (i.e. the shoulder opposite the arm that is in the sling). In the illustrated embodiment, the sling 10 also has two hand loops 16, one on either side of the pouch opening, configured for the patient's thumb to be placed on one side of one of the loops and four fingers to be placed on the other side, to prevent relative movement between the sling 10 and the arm (not illustrated in FIG. 1). The hand loops 16 and further features of the illustrated sling are described in detail in the above-mentioned patents and will be omitted here. The sling 10 has a length of stretchable loop tape 18 attached to the outside of each panel 12 of the pouch. The loop tape 18 may alternatively only be provided on one of the two panels 12. However, because the Ultimate Arm Sling® is ambidextrous, it is advantageous to provide the loop tape on both panels, so the system as a whole is ambidextrous. It will be appreciated based on a review of the above-mentioned patents that the loop tape 18 is advantageously made of a stretchable elastic material, to keep the integrity of the stretch-to-fit Ultimate Arm Sling® intact.

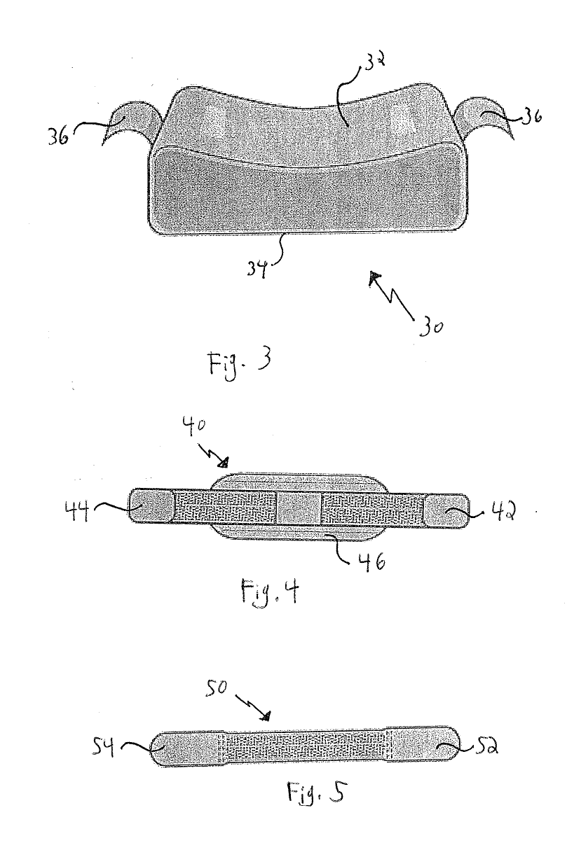

FIG. 2 illustrates a waist band 20. The waist band 20 has a back portion 22, to extend around the patient's back, and a relatively wider front portion 24, to extend around the patient's stomach, both made of a material that acts as a loop fastener. In other words, in a presently preferred embodiment, the entire soft fabric of the waist band (both the back portion 22 and the front portion 24) is made of loop material that adheres to hook. The front and back portions are connected at an adjustable fastener 26 and a buckle 28. Both fasteners 26, 28 may be adjustable; one fastener 26 may be adjustable and the other 28 may be fixed; or the band 20 may consist of a single piece of material joined only with one adjustable or non-adjustable buckle. In the illustrated embodiment, the buckle 28 includes a piece of narrower hook fabric looped through a buckle and attachable to the loop fabric of the front portion 24 of the waist band 20. The adjustable fastener 26 includes any desired combination of buckles to appropriately adjust the size of the waist band 20. In the illustrated embodiment, the adjustable fastener 26 needs to be sized only once, and does not come apart to open the waist band 20. The simpler buckle 28 can then be attached and detached for daily use, without having to resize the adjustable fastener 26. Furthermore, a length of hook tape 29 is attached to the front portion 24 of the waist band 20.

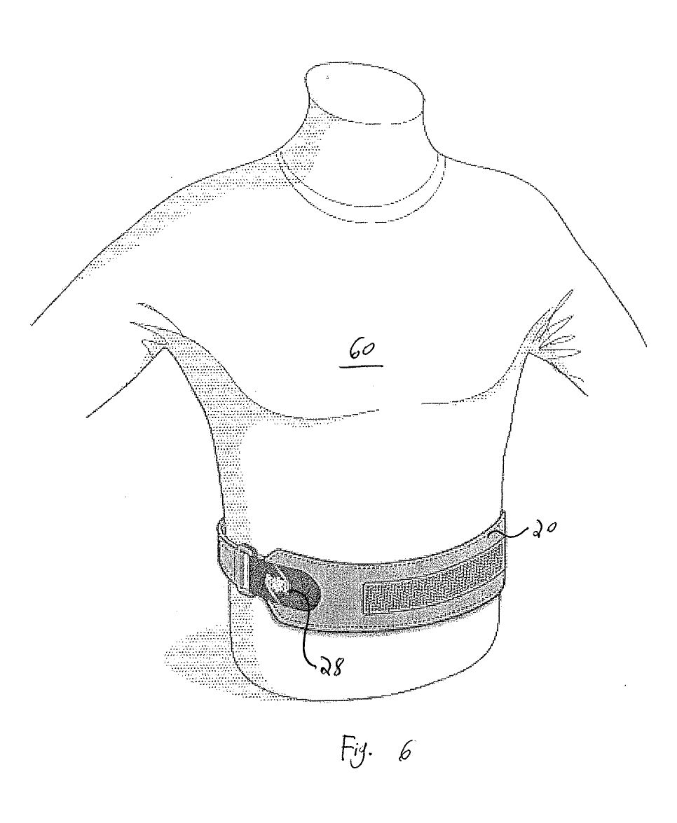

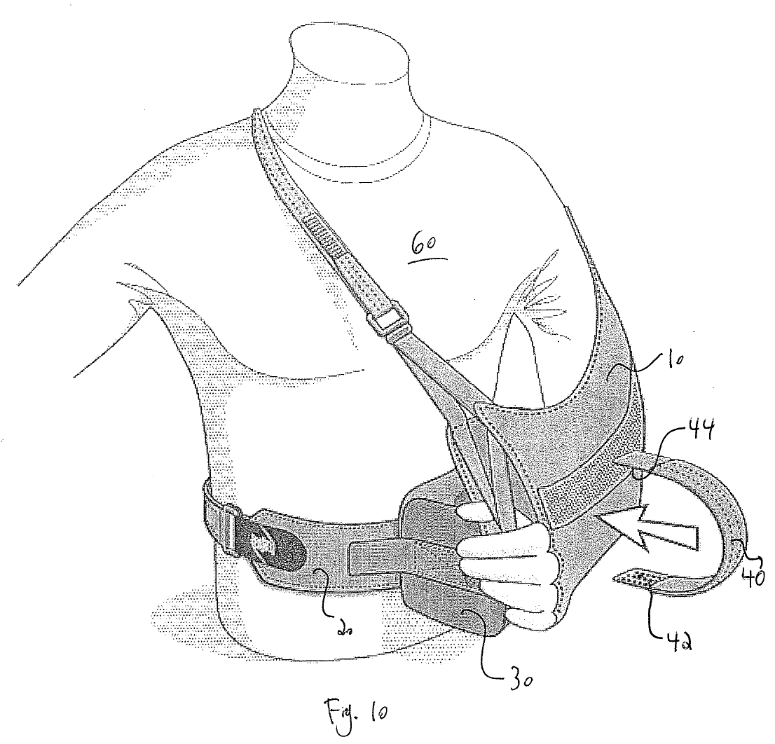

FIG. 3 illustrates a pillow 30. The pillow 30 may have any desired ergonomic shape to fit comfortably around the patient's waist at its inner surface 32, and is substantially flat at its outer surface 34. In a presently preferred embodiment, the pillow 30 is a foam core 15% to 25% abduction pillow made of a fabric that acts as a loop fastener. A length of hook tape 36 is provided at either end of the pillow 30 to attach to the loop fabric of the waist band 20, and another length of hook tape 38 (not visible in FIG. 3; see FIG. 8) is attached to the outer surface 34 of the pillow to engage the loop tape 18 on the sling 10.

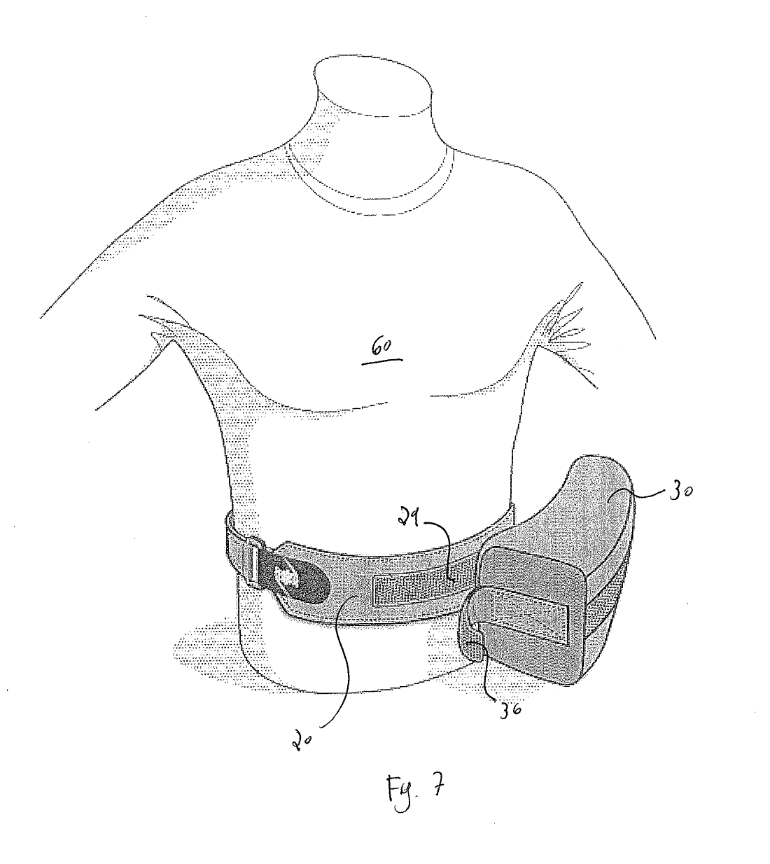

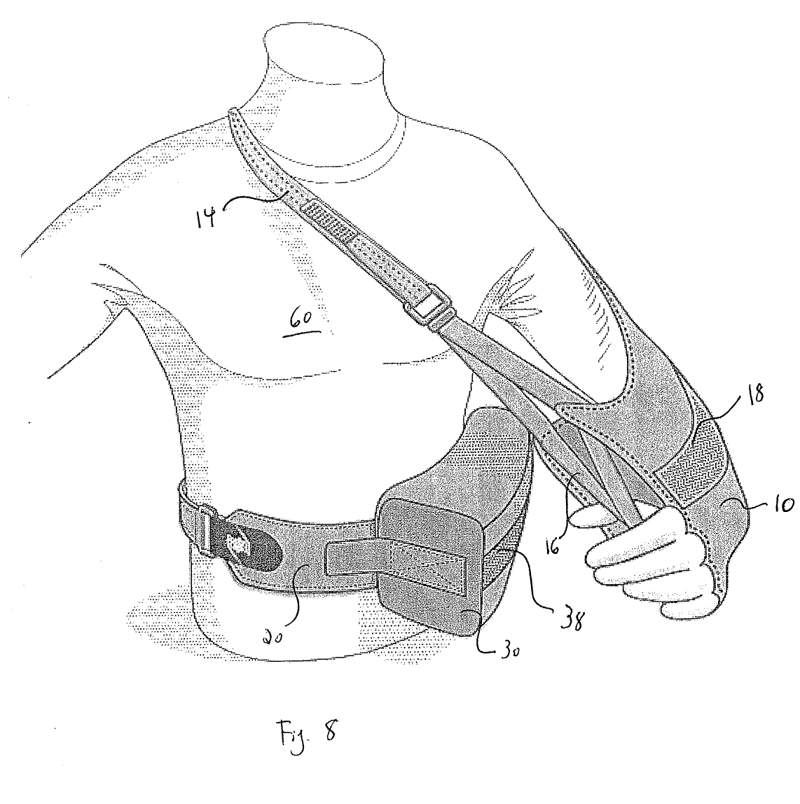

FIGS. 4 and 5 illustrate a forearm strap 40 and an elbow strap 50, respectively. Each of these straps 40, 50 has hook tape 42, 44, 52, 54 at either end. The forearm strap 40 has a wider center portion 46 for increased support.

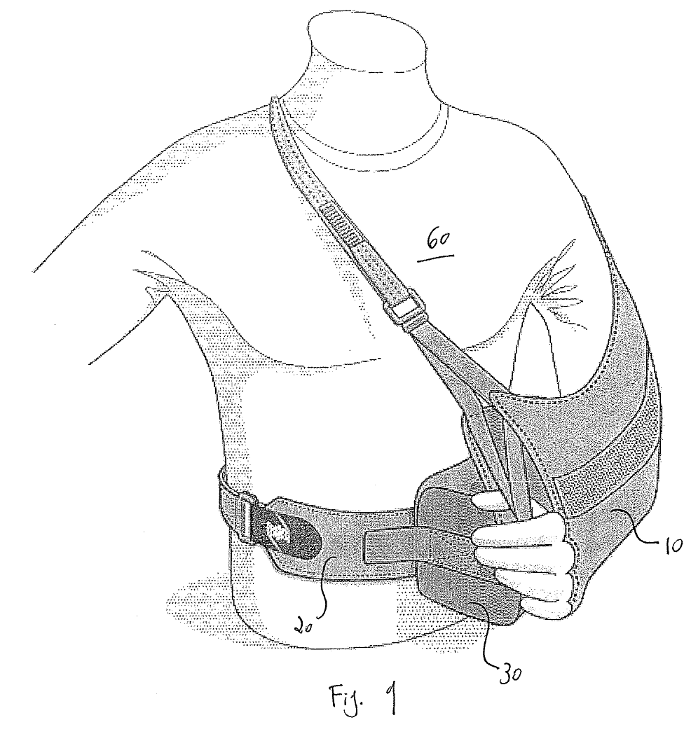

Turning now to FIGS. 6-14, a method of use of the inventive system will now be described. First, as shown in FIG. 6, a patient 60 dons the waist strap 20 using the buckle 28, by looping the hook tape through the buckle and adhering it to the loop fabric of the waist band 20. After this step, he or she has two alternative courses of action depending on whether or not the pillow 30 is to be used.

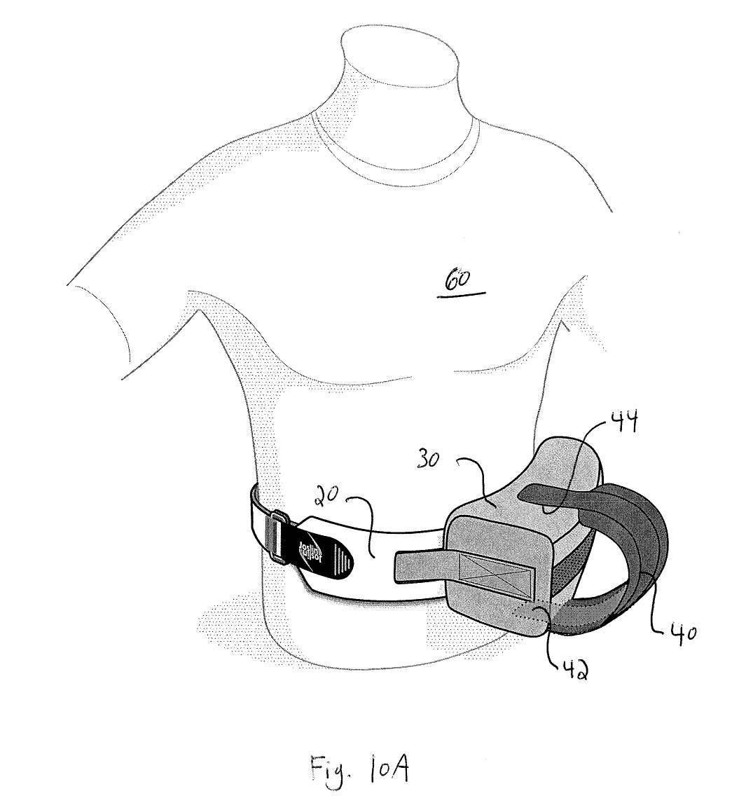

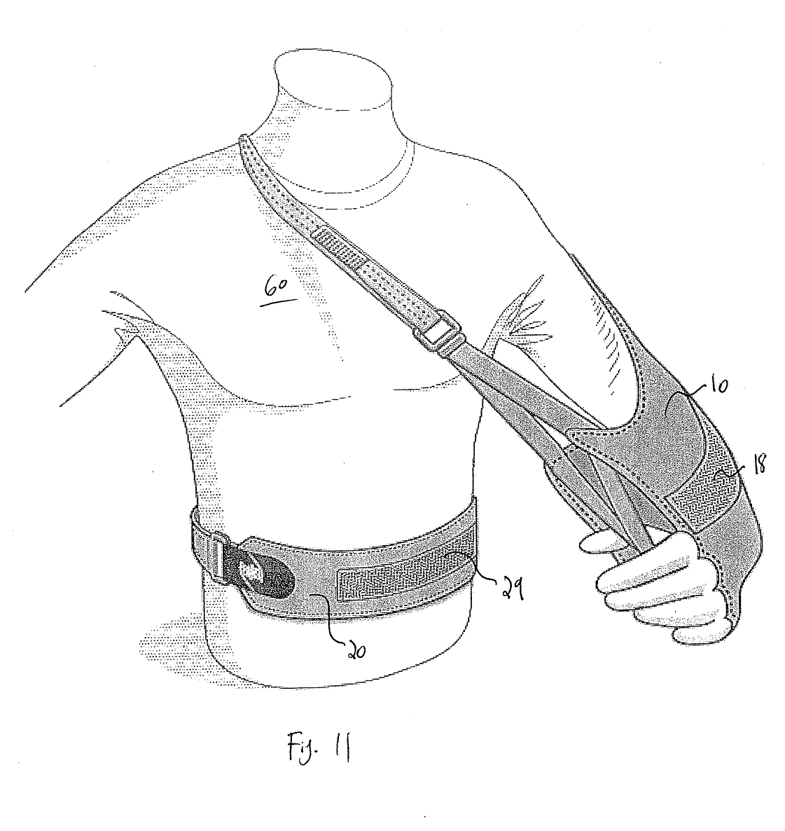

If the pillow is to be used, turning to FIG. 7, the pillow 30 is attached to the waist band 20 by applying the loop fabric of the pillow 30 to the hook tape 29 on the waist band 20, and by applying the hook tape 36 to the loop fabric of the waist band 20 at either side of the hook tape 29. Then, turning to FIG. 8, the patient dons the sling 10 by inserting his or her thumb and fingers through the hand loops 16 and hanging the strap 14 over his or her neck and non-treatment shoulder, as shown. The loop tape 18 on the panel 12 of the sling closest to the patient's body is applied to the hook tape 38 on the outer surface 34 of the pillow, leading to the arrangement seen in FIG. 9. Optionally, turning to FIG. 10, he or she can add the forearm strap 40 to further stabilize the arm, attaching the hook tape 42, 44 to the loop fabric at the top and bottom of the pillow 30 as seen in FIG. 10A (where the arm and sling have been removed for clarity). The patient can also optionally add the elbow strap by attaching the hook tape 52 to the loop tape 18 on the outermost panel 12 of the sling, and attaching the hook tape 54 to the loop fabric of the pillow 30 at the back end of the pillow 30. Thus, the arm is sufficiently stabilized in an abducted position, which is desirable for certain procedures or during certain times in the healing process.

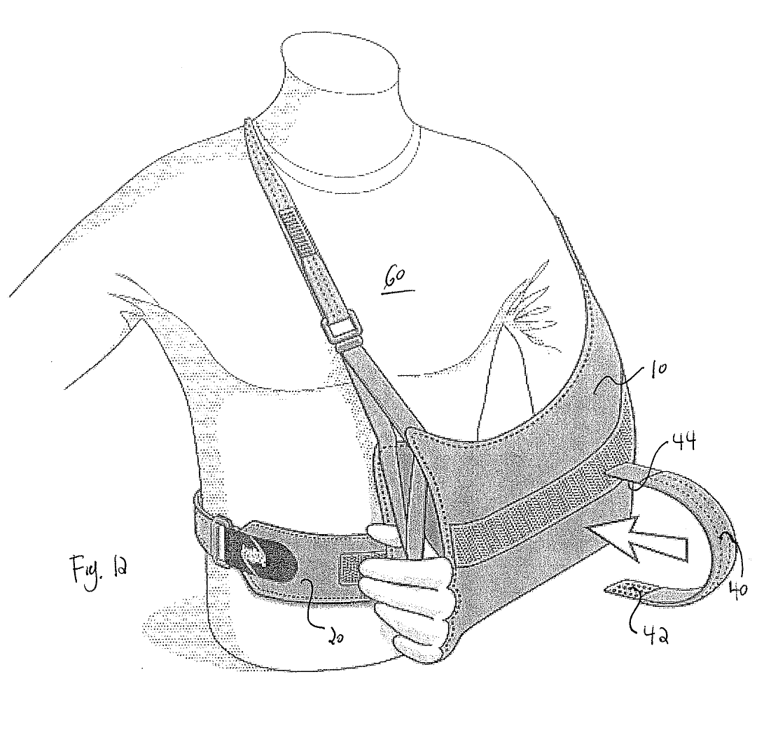

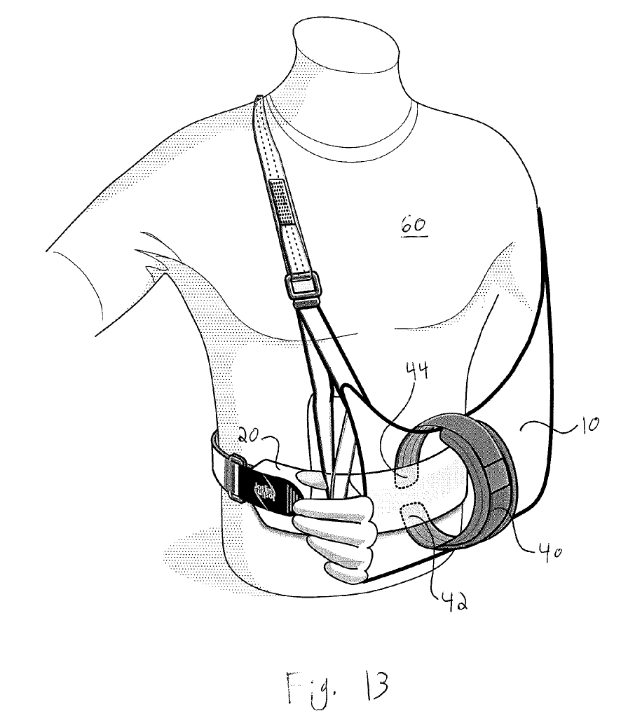

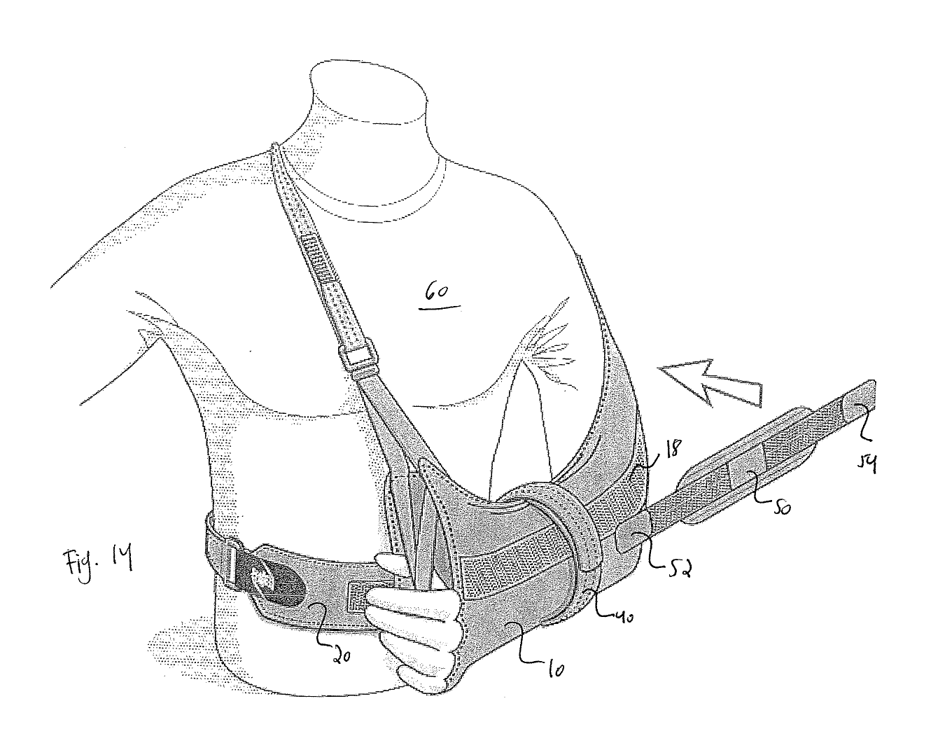

Alternatively, if the pillow is not to be used, such as for certain injuries, or once the pillow has been removed, such as at certain stages in the healing process, turning to FIG. 11, the patient dons the sling 10 without attaching the pillow 30 to the waist band 20. The loop tape 18 on the panel 12 of the sling closest to the patient's body is applied to the hook tape 29 on the waist band 20, leading to the arrangement seen in FIG. 12. Optionally, as seen in FIGS. 12 and 13, the forearm strap 40 can be added to further stabilize the arm, by attaching the hook tape 42, 44 to the loop fabric on the inside of the waist band 20. Additionally or alternatively, turning to FIG. 14, the elbow strap 50 can optionally be added. The hook tape 52 of the elbow strap 50 is applied to the loop tape 18 of the sling 10 on the outermost panel 12, and the hook tape 54 of the elbow strap 50 is applied to the loop fabric of the waist band 20 behind the hook tape 29. Thus, as is illustrated in FIG. 14, the arm is sufficiently stabilized in an non-abducted position, which is desirable for certain other procedures or during certain other times in the healing process.

Therefore, select components of the disclosed kit can be used for a variety of medical procedures or throughout the duration of a healing process, when the arm should be stabilized at a variety of positions.

As will be appreciated by those of ordinary skill in the art, the exemplary system described herein provides several advantages over other known systems:

1. stretchable loop strips sewn to each side of the Ultimate Arm Sling® both accommodate adherence to the hook of a Veltex® covered pillow, and maintain the integrity and comfort of the stretch-to-fit Ultimate Arm Sling®

2. a waistband which is usable with dual functions:
(A) arm abduction—the waistband provides hook adherence fabric to which the loop pillow fabric adheres (additional flaps on each end of the pillow offer further adherence support) and then to which the arm sling, with loop, adheres, and
(B) arm immobilization—the waistband provides the same type of adherence to the sling when the pillow is removed and the system becomes an arm immobilizer—not offered by any other company in the US.

3. a specially designed open and close “Joslin” tab within easy reach of the patient. Tabs are color coded according to size—green for average adult, orange for large adult sizes—the only opening in the waistband.

4. forearm and elbow straps for stabilizing the arm with or without the pillow.

As will be understood by those skilled in the art, the present invention may be embodied in other specific forms without departing from the essential characteristics thereof. Many other embodiments are possible without deviating from the spirit and scope of the invention. These other embodiments are intended to be included within the scope of the present invention, which is set forth in the following claims.

For example, while certain components have been described as being made of loop fabric, and others as having loop tape attached thereto, these embodiments are merely intended to be illustrative, and loop tape vs. loop fabric are interchangeable. It should furthermore be noted that the term “hook and loop fastener” in the context of the claims can refer to hook tape, hook fabric, loop tape, loop fabric, or any combination thereof. Both situations in which the fastener is integral to the relevant item (as in the case of, e.g., the pillow being made from loop fabric as in the described embodiments) as well as attached to it (as in the case of, e.g., the loop tape attached to the panels of sling as in the described embodiments) are within the scope of the claim language that a certain item comprises a fastener, where that fastener comprises a hook fastener or a loop fastener. Furthermore, the generic term “fastener” is not limited to hook and loop fasteners.

It should further be noted that this application as filed includes several FIGS. 10, 12, and 14) in which the forearm strap 40 and elbow strap 50 are reversed. In the presently preferred embodiment, the forearm strap 40 includes a wider portion 46 for increased support, and the elbow strap 50 does not include such a wider portion. This is clearly illustrated in FIGS. 4, 5, 10A, and 13.

What is claimed is:

1. A kit for supporting an arm of a patient having a torso, the arm, a first shoulder connected to the arm, and a second shoulder, the kit comprising:
a waist band configured to be worn around the torso and comprising a first fastener; and
a sling comprising two panels defining a pouch configured to support the arm, a strap attached to the panels and configured to be hung from the second shoulder, and a second fastener attached to at least one of the panels;
wherein the first and second fasteners are configured to be readily repeatedly fastened to and released from each other to releasably secure the sling to the waist band.

2. The kit of claim 1, wherein each of the fasteners comprises a hook fastener or a loop fastener.

3. The kit of claim 1, further comprising an additional strap configured to wrap around the sling, the arm, and the waist band, and to be readily repeatedly fastened and released to and from the wrapped position.

4. The kit of claim 3, wherein the additional strap comprises a first end with a third fastener and a second end with a fourth fastener, wherein the third and fourth fasteners are configured to be fastened to the waist band to secure the additional strap to the wrapped position.

5. The kit of claim 4, wherein each of the third and fourth fasteners comprises a hook fastener or a loop fastener.

6. The kit of claim 1, further comprising an additional strap comprising a first end configured to be readily repeatedly fastened and released to and from the sling, and a second end configured to be readily repeatedly fastened and released to and from the waist band, such that the additional strap wraps behind an elbow of the arm.

7. The kit of claim 6, wherein each of the first and second ends of the additional strap comprises a hook fastener or a loop fastener.

8. A kit for supporting an arm of a patient having a torso, the arm, a first shoulder connected to the arm, and a second shoulder, the kit comprising:
a pillow configured to be worn around the torso and comprising a first fastener; and
a sling comprising two panels defining a pouch configured to support the arm, a strap attached to the panels and configured to be hung from the second shoulder, and a second fastener attached to at least one of the panels;
wherein the first and second fasteners are configured to be readily repeatedly fastened to and released from each other to releasably secure the sling to the pillow, to thereby secure the arm at an abducted position relative to the torso.

9. The kit of claim 8, wherein each of the fasteners comprises a hook fastener or a loop fastener.

10. The kit of claim 8, further comprising an additional strap configured to wrap around the sling and the arm, and at least partway around the pillow, and to be readily repeatedly fastened and released to and from the wrapped position.

11. The kit of claim 10, wherein the additional strap comprises a first end with a third fastener and a second end with a fourth fastener, wherein the third and fourth fasteners are configured to be fastened to the pillow to secure the additional strap to the wrapped position.

12. The kit of claim 11, wherein each of the third and fourth fasteners comprises a hook fastener or a loop fastener.

13. The kit of claim 8, further comprising an additional strap comprising a first end configured to be readily repeatedly fastened and released to and from the sling, and a second end configured to be readily repeatedly fastened and released to and from the pillow or an additional garment, such that the additional strap wraps behind an elbow of the arm.

14. The kit of claim 13, wherein each of the first and second ends of the additional strap comprises a hook fastener or a loop fastener.

15. A kit for supporting an arm of a patient having a torso, the arm, a first shoulder connected to the arm, and a second shoulder, the kit comprising:
a waist band configured to be worn around the torso and comprising a first fastener;
a pillow comprising a second and a third fastener; and
a sling comprising two panels defining a pouch configured to support the arm, a strap attached to the panels and configured to be hung from the second shoulder, and a fourth fastener attached to at least one of the panels;
wherein the first and second fasteners are configured to be readily repeatedly fastened to and released from each other to releasably secure the pillow to the waist band;
and wherein the third and fourth fasteners are configured to be readily repeatedly fastened to and released from each other to releasably secure the sling to the pillow, to thereby secure the arm at an abducted position relative to the torso.

16. The kit of claim 15, wherein each of the fasteners comprises a hook fastener or a loop fastener.

17. The kit of claim 15, further comprising an additional strap configured to wrap around the sling and the arm, and at least partway around the pillow, and to be readily repeatedly fastened and released to and from the wrapped position.

18. The kit of claim 17, wherein the additional strap comprises a first end with a fifth fastener and a second end with a sixth fastener, wherein the fifth and sixth fasteners are configured to be fastened to the pillow to secure the additional strap to the wrapped position.

19. The kit of claim 18, wherein each of the fifth and sixth fasteners comprises a hook fastener or a loop fastener.

20. The kit of claim 15, further comprising an additional strap comprising a first end configured to be readily repeatedly fastened and released to and from the sling, and a second end configured to be readily repeatedly fastened and released to and from the pillow, such that the additional strap wraps behind an elbow of the arm.

21. The kit of claim 20, wherein each of the first and second ends of the additional strap comprises a hook fastener or a loop fastener.

22. The kit of claim 15, wherein the first and fourth fasteners are configured to be readily repeatedly fastened to and released from each other to releasably secure the sling to the waist band when the pillow is not present.

23. The kit of claim 22, wherein each of the fasteners comprises a hook fastener or a loop fastener.

24. The kit of claim 22, further comprising an additional strap,
wherein the additional strap is configured to wrap around the sling and the arm, and at least partway around the pillow, when the pillow is present;
wherein the additional strap is further configured to wrap around the sling, the arm, and the waist band, when the pillow is not present;
and wherein the additional strap is further configured to be readily repeatedly fastened and released to and from the wrapped positions.

25. The kit of claim 24, wherein the additional strap comprises a first end with a fifth fastener and a second end with a sixth fastener, wherein the fifth and sixth fasteners are configured to be fastened to the pillow or to the waist band to secure the additional strap to the wrapped position.

26. The kit of claim 25, wherein each of the fifth and sixth fasteners comprises a hook fastener or a loop fastener.

27. The kit of claim 22, further comprising an additional strap comprising:
a first end configured to be readily repeatedly fastened and released to and from the sling; and
a second end configured to be readily repeatedly fastened and released to and from:
the waist band, when the pillow is not present; and
the pillow, when the pillow is present;
such that the additional strap wraps behind an elbow of the arm, when the pillow is present and when the pillow is not present.

28. The kit of claim 27, wherein each of the first and second ends of the additional strap comprises a hook fastener or a loop fastener.
